(19) United States
(12) Patent Application Publication
(10) Pub. No.: US 2021/0252273 A1
(43) Pub. Date: Aug. 19, 2021

(54) TUBULAR PROPULSION DEVICES AND METHODS OF USE THEREOF

(71) Applicant: The Arizona Board of Regents on Behalf of the University of Arizona, Tucson, AZ (US)

(72) Inventor: Marvin J. Slepian, Tucson, AZ (US)

(21) Appl. No.: 17/251,374

(22) PCT Filed: Jun. 12, 2019
(86) PCT No.: PCT/US2019/036811

§ 371 Date: Dec. 11, 2020

Related U.S. Application Data
(60) Provisional application No. 62/684,130, filed on Jun. 12, 2018.

(51) Int. Cl. A61M 60/40; A61F 2/04

(52) U.S. Cl. CPC A61M 60/40; A61F 2/04; A61M 2205/0266; A61F 2002/048; A61M 2205/0294; A61M 2250/00; A61M 2205/0216

(57) ABSTRACT

Tubular propulsion devices and systems and methods for using such devices and systems to restore, replace, or augment or otherwise modulate active transport of fluids through a diseased or damaged tubular organ or organ segment are described. The devices have a hollow center surrounded by a peripheral wall. The devices can be multilayer devices. The devices may be single tube devices or multi-section devices. Typically, elements for altering the structure of the device, such as via compression, expansion, twisting, and/or contraction of one or more sections of the peripheral wall, are included in the walls or are outside or inside, of the walls of the device. The devices undergo intermittent change of the contained volume (luminal volume) in a sequential manner to direct fluid flow. In use, the devices are able to serve as local mini- or regional-pumps.

### CROSS-REFERENCE TO RELATED APPLICATION

The present application claims priority to U.S. Application No. 62/684,130, filed Jun. 12, 2018, the disclosure of which is incorporated herein in its entirety.

### FIELD OF THE INVENTION

This invention is generally in the field of devices to aid in the transport of fluids through vessels in the body, and methods for using such devices.

### BACKGROUND OF THE INVENTION

Higher animals, such as mammals or man, contain tubular organs and organ components, which maintain normal physiologic functions.

Tubular organs or organ components typically serve multiple generic functions. One function includes mass containment. This may include fluid containment in the case of blood, urine, bile or lymph; bulk containment in the form of digesting food, chyme or stool; gaseous containment in the form of air or other inhaled substances. A second function of tubular organs or components includes that of a secretory or modulatory function, typically by elements in the wall of the tubular organ, e.g. to modulate material contained within or traversing through. Thirdly, tubular organs and organ components may serve a transport function. From this functional perspective, they may similarly transport blood in the case of the circulatory system; urine in the case of the genitourinary system; food and stool in the case of the gastrointestinal system; lymph in the case of the lymphatic system; and air and gas in the case of the respiratory system.

However, with age, trauma, and disease many of tubular tissues become dysfunctional. A common form of malady for tubular structures includes progressive encroachment of the lumen by a variety of processes, which may initiate or involve intra-or endoluminal narrowing. Examples of common problems that can develop over time in tubular organs include the development of atherosclerosis in the coronary artery; wall thickening such as occurs with the infiltration of a tumor into the wall of the intestine with eventual luminal obstruction; or external encroachment or constriction, i.e. an ectoluminal process as occurs with an encircling mass or tumor. Pathologies include fibrosis, infiltrative/metastatic tumors, strictures, compressive external growths or other forms of stenoses. Conversely, tubular function may be compromised by enlargement, wall weakening, out-pouching, dilation, or frank- or pseudo- aneurysm formation, with the potential for rupture. These processes often hinder tubular transport function resulting in fluid buildup and backup, with both forward flow and backward often combined with reduced perfusion and transport, i.e. flow consequences, often affecting many organs, if not the body as a whole. This can result in significant illness and dysfunction for the afflicted animal, including humans.

While endotubular technologies have recently developed, these devices focus on restoring anatomic geometries and luminal architecture to allow return of the containment function to the hollow or tubular organ. These devices typically include constructs such as stents, temporary splinting or stenting catheters, and/or endografts and endoluminal paving systems, such as described in U.S. Pat. No. 6,290,729 to Slepian and Massia; U.S. Pat. Nos. 5,634,946 and 5,213,580 to Marvin J. Slepian. However, these devices have not improved the active transport function of tubular organs.

There is a need for improved devices and materials that can aid in the active transport of fluids through tubular organs and/or organ segments in which their active transport function has been compromised.

Therefore, it is an object of the invention to provide devices and systems that can aid in the active transport of fluids through tubular organs and organ segments.

It is a further object of the invention to provide methods of using such devices and systems to facilitate active transport of fluids through tubular organs and organ segments.

### SUMMARY OF THE INVENTION

Tubular propulsion devices and systems and methods for using such devices and systems to restore, replace, or augment, or otherwise modulate active transport of fluids through a diseased or damaged tubular organ or organ segment are described herein. The devices have a hollow center surrounded by a peripheral wall. The tubular propulsion devices may be single tube devices or multi-section tubular devices.

Typically, elements for altering the structure of the device, such as via compression, expansion, twisting, and/or contraction of one or more sections of the peripheral wall, are included in the walls or are outside, or optionally inside, of the walls of the device. The walls can include one or more openings, such as flaps, that can be actuated to push or propel the fluid through the device.

Generally, a single tube device has one or more elements for altering the structure of the device positioned along the length of the device. A multi-section device typically includes an assembly of a plurality of sections aligned and connected to each other such that together they form the tubular device, where each section has a hollow center surrounded by a peripheral wall containing one or more elements for altering the structure of the device.

The tubular device can be a single layer or multi-layer device. For multi-layer devices, optionally the outer wall of the device is stiffer than the element(s) (e.g. a balloon) inside the device. This allows the inner elements to temporarily expand and create a temporary reduction in lumen volume for a portion of the wall of the tubular device; followed by contraction of the inner elements, allowing the volume of the lumen to increase to its initial volume and thereby push a fluid through the device. Optionally, the inner element(s) move in a unidirectional manner along the length of the device, which corresponds with the desired direction of fluid flow.

In use, the devices are able to serve as local mini- or regional-pumps, optionally creating a pulsatile fluid movement through the device. The device is exposed to a repeating cycle of exposure to one or more stimuli that actuate the materials or elements inside the walls of the device to alter the configuration of the tubular structure for a suitable time period, followed by a removal of the stimulus for a suitable time period during which the device returns to its original state to facilitate active transport of fluids through the devices.

The devices may be controlled by a controller that communicates with the device via the one or more interconnects connecting different sections of the peripheral wall in a single tube device or different sections of the device in the multi-section device. The devices may be in electrical communication with one or more intimate controllers that are implanted or inserted in the patient's body, and, optionally, one or more external controllers. Typically, the one or more intimate controllers are in communication with one or more of the interconnects, optionally with all of the interconnects, in the device. The one or more intimate controllers may control the sequence, direction, and/or rate of actuation of one or more, optionally all, of the sections of the device. The intimate controllers may be in communication with the external controller, if present, to provide an overall control unit to control the sequence, direction, and rate of actuation. The external controller may be used to turn on, turn-off, monitor, adjust, and/or to control the sequence, direction, and/or rate of actuation of one or more, optionally all, of the sections of the device.

### BRIEF DESCRIPTION OF THE DRAWINGS

FIGS. 1A-1E are illustrations of a tubular propulsion device in its resting state (FIG. 1A) and four different configurations of the device in its actuated state using compression of one or more walls (FIGS. 1B-1E). FIG. 1B shows only one portion of the wall compressed. FIG. 1C shows two portions of the wall compressed, where one portion is located opposite the other portion. FIG. 1D shows three portions of the wall compressed, where two portions are located opposite each other and the remaining third portion is located in a plane that is substantially perpendicular to a plane connecting the first and second portions. FIG. 1E shows four portions of the wall compressed, where two portions are located opposite each other with a first plane connecting the first and second portion, and the remaining other two portions are located opposite each other, with a second plane connecting the third and fourth portions, where the first and second planes are substantially perpendicular to each other.

FIGS. 2A-2B are illustrations of tubular propulsion devices that push a fluid through the device via twisting actions. FIG. 2A shows the tubular propulsion device in its resting state and in its actuated state with one twist in the walls of the device. FIG. 2B shows the tubular propulsion device in its resting state and in its actuated state with multiple twists in the walls of the device.

FIGS. 3A-3B are illustrations of tubular propulsion devices that push a fluid through the device via contractions in the walls. FIG. 3A shows the tubular propulsion device in its resting state and in its actuated state with one contraction in the walls of the device. FIG. 3B shows the tubular propulsion device in its resting state and in its actuated state with multiple contractions in different sections of the peripheral wall of the device.

FIGS. 4A-4E are cross-sectional views of the devices illustrated in FIGS. 1A-1E comparing the cross-section of the device in its resting state (FIG. 4A) to different cross-sections of the device in its actuated state (FIGS. 4B-4E). FIG. 4B shows only one portion (a top portion) of the wall compressed thereby constricting the lumen in the device. FIG. 4C shows two portions of the wall (a top portion and an opposing bottom portion) compressed thereby constricting the lumen in the device. FIG. 4D shows three portions of the wall (a top, a bottom, and a first side portion) compressed thereby constricting the lumen in the device. FIG. 4E shows four portions of the wall (a top, a bottom, a first side and a second side portion) compressed thereby constricting the lumen in the device.

FIGS. SA-SC are schematics of three different methods for actuating the peripheral walls of tubular devices. FIG. 5A shows the peripheral wall of the tubular device compressed via an external actuator. FIG. 5B shows a tubular device inside of a tubular organ, where the peripheral wall of the device is compressed via one or more elements in the walls of the device itself. FIG. 5C shows a tubular device where the peripheral wall contains flaps that are actuated to push a fluid through the device.

FIGS. 6A-6C are illustrations showing embodiments of multi-section tubular propulsion devices. FIG. 6A demonstrates an assembled multi-section device with “in-series”, or tandem, arrangement of sections. FIGS. 6B and 6C illustrate two different embodiments of the multi-section device in their resting and actuated states, showing a defined sequence of actuation of each section in the device. Each section operates independently. The sequential actuation of the sections in the device may be configured to provide a uni-directional movement of the fluid in the lumen and directional lumen volume displacement of the fluid through the lumen of the device.

FIGS. 7A-7C are cross-sectional views of sections for exemplary multi-section devices showing several embodiments of the section actuation. In FIG. 7A an inner element of the wall of the section moves inwardly. FIG. 7B illustrates a section with a plurality of elements in the wall, and alternating elements, such as located at intervals of 180°, or other intervals, such as 30°, 45°, 90°, etc, that are able to sequentially move radially inward to create an activation scheme displacing different zones of fluid content in the lumen. FIG. 7C illustrates an embodiment of a section where the outer wall moves radially inward. In this embodiment, one portion of the peripheral wall of the section is compressed. Similar to embodiments in FIGS. 4C-4E, each section may have two, three, or four, or more, moving portions of the walls, and actuate by moving in, or compressing, one or more portions of the wall in the section.

FIGS. 8A-8C are illustrations of several exemplary embodiments of system containing a tubular device, such as a multi-section device, with one or more controllers. FIG. 8A shows a connection between sections in the multi-section device. These may be direct communication connections. FIGS. 8B and 8C show an implantable controller in contact with two sections (FIG. 8B) or with a plurality of sections, such as an entire device (FIG. 8C) to control the sequence, direction and/or rate of actuation. FIG. 8C also illustrates an external controller, which is located outside of the body, in communication with the implantable controller, located inside the patient's body, which together form an overall control unit to control the sequence, direction and/or rate of actuation of the device.

FIG. 9 is an illustration of a peripheral wall and a portion of a peripheral wall. FIG. 9 shows a cross-section of a tubular device where a portion 80 of the peripheral wall 82 is defined by an arc between two radii 84 and 86 positioned at about 90° to each other.

### DETAILED DESCRIPTION OF THE INVENTION

Tubular propulsion devices and systems which may be placed within diseased tubular organs or organ components or tubular organs or organ components in which their structure has been modified in a manner that hinders local transport function (typically due to a disease or disorder) are described herein. The devices and systems can restore, replace or augment, or otherwise modulate the local transport function of the organ or organ component in which they are placed.

Generally, the tubular organ to be treated is one which locally imparts the transport function. For example, the tubular organ to be treated can be the ureter. In the genitourinary system the kidney is largely passive, filtering urine, providing a fluid and pressure head to the ureter, which has contained pulsatile and propulsive elements moving urine outward for elimination. Other tubular organs in which the device(s) or system(s) can be placed include the gastrointestinal tract. In the gastrointestinal tract, contained motor function in the gut wall, in the form of peristalsis movement and undulation, is the primary mechanism by which food and its digestion and degradation products move through the gastrointestinal system. This is in contrast to the circulatory system where blood vessels, while having some dynamism, such as in the form of spasm and constriction, are more passive in the sense that the heart provides the propulsive force moving blood throughout the more static circulatory system.

The devices are typically implanted in a subject with a tubular organ in need of treatment. The subject may be a vertebrate, more specifically a mammal (e.g., a human, horse, pig, rabbit, dog, sheep, goat, non-human primate, cow, cat, guinea pig or rodent), a fish, a bird or a reptile or an amphibian. The subject may be a human or a veterinary animal.

### I. Devices and Systems

The tubular devices and systems can be modified as needed to serve different organs, disease states, or as a given biological or clinical situation dictates. The devices can have a variety of structures, with varying thicknesses, lengths, and three-dimensional geometries. Further, the devices can have one layer or multiple layer configurations. The devices may be single tube devices or multi-section devices.

A system can include one or more tubular devices, and a controller. The tubular devices typically include one or more interconnects. The controller can be located inside the patient's body, e.g. in intimate connection with the tubular device(s), or external to the patient's body, i.e. outside the body. Optionally, the system includes both a controller located inside the patient's body and an external controller, which are in electronic communication with each other and together form an overall control unit to control the sequence, direction and/or rate of actuation of the tubular device.

A. Structure of Single Tube Devices

The tubular propulsion devices have a hollow center, or lumen, surrounded by a peripheral wall. The peripheral wall is formed of an outer surface and an inner surface, which defines the lumen of the device. Typically, elements for altering the structure of the device, such as via compression, twisting, and/or contraction of one or more portions of the peripheral walls, are included in the walls or are outside of the walls of the device. Optionally, the walls include one or more openings, such as flaps, or contained wall components or structures that move or change shape (expand or contract) in a controlled manner and thereby aid in pushing the fluid through the device.

The wall of the device may be multi-layered—with surrounding concentric wall structures. Contained within a given wall layer or between layers, propulsive elements may be contained. For example, interspersed between a layer may be magnets in the form of small shapes, such as prisms, spheres and the like which may be actuated leading to opposite wall attraction, alternating with repulsion resulting in a pulsatile wave. If the actuation is performed sequentially along the length of the device, it this can lead to net directional fluid movement. In another embodiment, contained bladder, bladders or extendable materials, such as balloons, may be contained. Bladders may contain a fluid, gas, ferrofluid, magnetic slurry, or other means to allow the bladder to expand and contract in a controlled manner, resulting in a sequential and directional change of geometry to occur to the device and in sequential volume change with net directional fluid movement through the device.

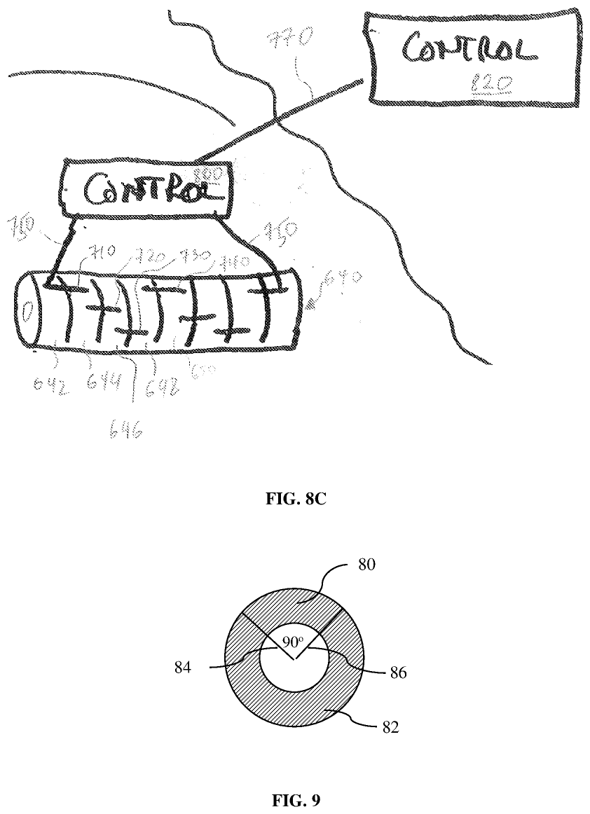

A portion of the peripheral wall can contain one or more elements or multiple portions of the peripheral wall can contain elements that respond to a stimulus to actuate the device or that portion of the device. A portion of the wall refers to less than the entire wall. With respect to tubular devices having a circular cross-section, a portion of the wall refers to a segment of the peripheral wall that, in the cross-section, is defined by an arc between two radii positioned less than 360° to each other, typically less than 180°, optionally about 90° or less, such as about 80° or less, about 70° or less, about 60° or less, about 50° or less, about 45° or less, about 40° or less, about 30° or less to each other, optionally about 90° to about 5°, about 80° to about 5°, about 70° to about 5°, about 45° to about 5°, about 30° to about 10°, about 30° to about 5°, about 20° to about 10°, about 20° to about 5°, or about 15° to about 5° to each other. An exemplary portion of the peripheral wall of the device is shown in FIG. 9. FIG. 9 shows a cross-section of a tubular device where a portion 80 of the peripheral wall 82 is defined by an arc between two radii 84 and 86 positioned at about 90° to each other.

Along the length of the device, the portion of the peripheral wall may be continuous along a straight line and have a length of the entire length of the device, or less than the entire length of the device, such as about 90%, about 80%, about 70%, about 60%, about 50%, about 40%, about 30%, about 20%, about 10%, or about 5% the length of the device. A portion of the peripheral wall may have a length between about 90% and about 5%, about 80% and about 5%, about 70% and about 5%, about 45% and about 5%, about 30% and about 10%, about 30% and about 5%, about 20% and about 10%, about 20% and about 5%, or about 15% and about 5% the length of the device.

a. Hollow with any Cross-Sectional Shape

Typical shapes for the tubular propulsion device include hollow cylinders with a circular cross-section. The devices can be formed of a plurality of sections that align and attach to each other, to form a peripheral wall with a hollow lumen. The devices may have other hollow prism shapes, such as cuboid or polygonal structures with multiple sides, such as triangular prism, hexagonal prism, heptagonal prism, octagonal prism, etc. with a hollow lumen and with adequate length for a given use. The devices may also be oval or ellipsoidal or otherwise configured to approximate the natural or therapeutically desired geometry for the site of implantation.

Typically, the outer diameter of the tubular devices is between about 1 mm and about 40 mm, such as between about 1 mm and about 30 mm, between about 1 mm and about 20 mm, between about 1 mm and about 10 mm. The length of the devices may be variable and depend on the length of the insertion site, or the length of the region of the body lumen needed support in structure and and function. For example, the length of the device may be between 2 mm and 80 mm, between 2 mm and 70 mm, between 2 mm and 60 mm, between 2 mm and 50 mm, between 2 mm and 40 mm, between 2 mm and 30 mm, between 2 mm and 20 mm, or between 2 mm and 50 mm.

The inner diameter of the tubular devices (the lumen diameter) in the resting state may be between about 1 mm and about 35 mm, between about 1 mm and about 25 mm, about 1 mm and about 15 mm, about 1 mm and about 8 mm, about 1 mm and about 5 mm, about 1 mm and about 4 mm, about 1 mm and about 3 mm, about 1 mm and about 2 mm. The lumen diameter may be altered by the elements that alter the configuration of tubular structure of the devices. The lumen circumference in a cross-section of the device, when the device is actuated, may be reduced by a percentage between 10% and 100%, such as by about 10%, about 20%, about 3%, about 40%, about 50%, about 60%, about 70%, about 80%, about 90%, about 100% compared to the circumference of the cross-section of the device in the resting state.

Overall, the smallest dimensions of the device are typically 1 mm or greater than 1 mm. The dimensions of the device may be governed by the use of the device for supporting the anatomy and function of given tubular organ. Examples of tubular organs include the cardiovascular system including the heart and blood vessels; the alimentary tract including the esophagus stomach small and large intestine; the respiratory tree including trachea, bronchi and alveoli; the lymphatic system; the genitourinary system with the hollow kidney, ureter, bladder, and urethra; the reproductive system, including the fallopian tubes, uterus or spermatic ducts; various glandular organs, including endocrine and exocrine tissues with ducts. The devices for the use in the cardiovascular system may have an outer diameter between about 1 mm and about 30 mm. The devices for the use in the alimentary tract may have an outer diameter between about 5 mm and about 40 mm. The devices for the use in the respiratory tree may have an outer diameter between about 1 mm and about 40 mm. The devices for the use in the lymphatic system may have an outer diameter between about 1 mm and about 15 mm. The devices for the use in the genitourinary system may have an outer diameter between about 1 mm and about 20 mm. The devices for the use in the reproductive system may have an outer diameter between about 1 mm and about 30 mm. The devices for the use in the glandular organs may have an outer diameter between about 1 mm and about 5 mm.

b. Walls of the Devices

The walls of the hollow devices may have a variety of different structures, such as solid, partially perforated or contain large gaps. Examples of suitable devices include slotted tubes and braided tubes with a range of porosities, such as from 100 nm up to 15 mm, from 500 nm to 15 mm, from 100 nm to 1 mm, from 1 mm to 15 mm, from 1 mm to 10 mm, and from 5 mm to 15 mm. To determine the suitable pore size, this is may be scaled to the actual dimensions of the construct. A desired percent open surface area (i.e. percent wall openness) may be designed into the walls of the device to allow transmural exchange of nutrients, fluid and cells necessary for maintaining the health of the native tubular structure into which the device is implanted. For example, if placed in a blood vessel, a non-porous structure could lead to the withering of the underlying tissue wall. Thus, so a defined percent (%) porosity may be imparted in the walls of the device to maintain the health of the surrounding tissue. For devices that are designed to be placed in fibrotic tissue or otherwise non- actively metabolizing tissue or as a connected structure acting as a bypass, the walls of these devices can be nonporous.

The porosity of the walls can be expressed as percent wall openness, such as ranging from 0% for a non- porous structure to up to 95% porosity for a maximally porous structure, while maintaining adequate structural integrity. Optionally, the porosity of the walls ranges from 1% to 10% for minimally porous walls, from greater than 10% up to 50% for walls with medium porosity, and from 50% to 95%, for walls with high levels of porosity.

The peripheral walls can have a smooth, patterned, or rough surface. For example, the walls may be processed prior to insertion with either light-induced or chemical etching, pitting, slitting, or perforation depending upon the application. In addition, the shape of any micro (10 nm to 1 μm) or macro (>1 μm to 4.0 mm) perforation may be further geometrically modified to provide various surface areas on the inner versus outer seal surface. The surfaces of the walls of the device may be further modified with bound, coated, or otherwise applied agents, e.g cyanoacrylates or adhesives such as those derived from fungal spores, sea mussel (e.g. polymers containing 3,4-dihydroxyphenylalanine (DOPA) and/or related catechols) or fibrinogen.

The walls of the device may include perforations or pores. By using a fragmented tubular polymer surface with corresponding expansions along predicted perforations, a significant mechanical stability is provided to the device. In addition, the amount of foreign material placed within the vessel is minimized.

The wall thickness of the device when in the resting state and measured along the radius of the cross-section of the device from the outer edge of the device to the lumen may be between about 1 mm and about 9 mm, between about 1 mm and about 8 mm, about 1 mm and about 7 mm, about 1 mm and about 6 mm, about 1 mm and about 5 mm, about 1 mm and about 4 mm, about 1 mm and about 3 mm, about 1 mm and about 2 mm.

i. Wall Sections

The walls may contain one or more sections with different properties. For example, one section of the wall may be thinner than another section of the wall. Different sections of the walls may be formed from different materials, such as different polymers. Different sections of the walls may contain different elements to actuate the particular portion of the wall.

In this manner, a first section of the wall is actuated by a first stimulus, while the other sections remain stationary, then the first section will return to its initial, resting position, and another section, typically, an adjacent section, optionally one located upstream in the direction of the flow of the fluid, becomes actuated. Next, the actuated section returns to its resting state, and the adjacent section becomes actuated. This process can be repeated multiple times to push a fluid in a unidirectional manner through the organ or organ segment. A similar process can be used to move the fluid in two directions, if needed (e.g. a first direction and in the opposite direction).

Optionally, all of the sections are actuated at the same time. Alternatively, different sections are actuated at different times, and/or via different stimuli.

Optionally, the propulsive movement may lead, upon relaxation to a net void or vacuum also facilitating ingress or directional movement of the luminal fluid through the device.

(a) Structure of Multi-Section Propulsion Devices

The tubular propulsion device may be a multi-section propulsion device. Generally, the multi-section propulsion device is an assembly of a plurality of sections arranged in series, where each section has a hollow center surrounded by a peripheral wall. Typically, the peripheral wall of each section includes one or more elements for altering the structure of the device, such as via compression, expansion, twisting, and/or contraction of the peripheral wall. The one or more elements may be included in the walls or are outside, or optionally inside and outside of the peripheral wall. The peripheral wall of each section can include one or more openings, such as flaps, that can be actuated to push or propel the fluid through the device.

The multi-section propulsion device may include two or more sections in any arrangement suitable to serve as local mini- or regional-pumps. The multi-section propulsion device may have the sections arranged to form a shape matching the anatomical shape of the tubular organ or organ segment receiving the device. For example, the multi-section propulsion device may be an assembly of two or more sections forming the linear device. The multi-section propulsion device may be a semi-circular assembly of two or more sections forming a semi-circular device. The multi-section propulsion device may be an angled or L-shaped assembly of two or more sections forming an angled or an L-shaped device. The multi-section propulsion device may be a plurality of semi-circular assemblies forming a snaked or wave-shaped device.

Generally, each section in the multi-section propulsion device operates independently of the neighboring section. The sections in the device are typically arranged to have sequential order for sequentially altering the structure of the sections in the device. This sequential actuation of the sections in the device may be configured to provide a uni-directional movement of the lumen content. The sequential actuation of the sections in the device may be configured to provide a bi-directional movement of the lumen content. Multi-section devices may provide a uni-directional movement of the lumen content. Multi-section devices may provide a bi-directional movement of the lumen content. Multi-section devices may provide a uni-directional or bi-directional movement of the fluid in the lumen.

Generally, the sections in the multi-section propulsion devices have a structure of the walls and cross-sectional shapes and actuation as described above for the single tube devices.

ii. Materials

The walls of the devices can be formed of any flexible, biocompatible materials, such as metals, plastics, rubber, or tissue materials, or combinations thereof. The elements that induce a change in shape of the wall can be included in the wall or one or more sections thereof. Alternatively, they can be outside of the device.

Exemplary materials for forming the walls of the device include metals such as titanium, nitinol and surgical stainless steel, ceramics, and synthetic polymeric materials. Non-biodegradable materials include polyethylene, TEFLON™ and pyrolytic carbon. Preferred biodegradable polymers include polylactic acid, polyglycolic acid, polycaptolactone and copolymers thereof.

(a) Polymeric Materials

Suitable polymeric materials for forming the walls of the device include both biodegradable and biostable polymers and copolymers.

The polymers that form that walls or one or more sections of the walls can be polymers or copolymers of carboxylic acids such as glycolic acid and lactic acid, polyalkylsulfones, polycarbonate polymers and copolymers, polyhydroxybutyrates, polyhydroxyvalerates and their copolymers, polyurethanes, polyesters such as poly(ethylene terephthalate), polyamides such as nylons, polyacrylonitriles, polyphosphazenes, polylactones such as polycaprolactone, polyanhydrides such as poly[bis(p-carboxyphenoxy)propane anhydride] and other polymers or copolymers such as polyethylenes, hydrocarbon copolymers, polypropylenes, polyvinylchlorides and ethylene vinyl acetates.

Representative synthetic polymers are: poly(hydroxy acids) such as poly(lactic acid), poly(glycolic acid), and poly(lactic acid-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(N-isopropylacrylamide, variously abbreviated PNIPA, PNIPAAm, NIPA, PNIPAA or PNIPAm), poly(isobutyl acrylate), and poly(octadecyl acrylate) (jointly referred to herein as “polyacrylic acids”), poly(butyric acid), poly(valeric acid), and poly(lactide-coaprolactone), copolymers and blends thereof. As used herein, “derivatives” include polymers having substitutions, additions of chemical groups, for example, alkyl, alkylene, hydroxylations, oxidations, and other modifications routinely made by those skilled in the art.

Representative natural polymers include proteins, such as zein, modified zein, casein, gelatin, gluten, serum albumin, or collagen, and polysaccharides, such as cellulose, dextrans, hyaluronic acid, polymers of acrylic and methacrylic esters and alginic acid. Synthetically modified natural polymers include alkyl celluloses, hydroxyalkyl celluloses, cellulose ethers, cellulose esters, and nitrocelluloses, acrylic or methacrylic esters of above natural polymers to introduce unsaturation into the biopolymers.

Examples of non-biodegradable polymers include ethylene vinyl acetate, poly(meth)acrylic acid, polyamides, copolymers and mixtures thereof. These polymers can be obtained from sources such as Sigma Chemical Co., St. Louis, Mo., Polysciences, Warrenton, Pa., Aldrich, Milwaukee, Wis., Fluka, Ronkonkoma, N.Y., and BioRad, Richmond, Calif. or else synthesized from monomers obtained from these suppliers using standard techniques.

(b) Biodegradable Polymers

The walls or one or more sections of the wall may be formed from biodegradable materials, such as biodegradable polymers. In general, the biodegradable materials degrade either by enzymatic hydrolysis or exposure to water in vivo, or by surface or bulk erosion.

Examples of biodegradable polymers include polymers of hydroxyacids such as lactic acid and glycolic acid, and copolymers with PEG, polyanhydrides, poly(ortho)esters, polycaprolactones, polyurethanes, poly(butyric acid), poly(valeric acid), poly(lactide-coaprolactone), blends and copolymers thereof.

Biodegradable polymers can be selected with specific degradation characteristics to provide a material having a sufficient lifespan for the particular application. Biodegradable polymers includes polymers, copolymers, and blends of polymers that are non-permanent and removed by natural or imposed therapeutic biological and/or chemical processes. As such, bioerodable or bioabsorbable polymers and the like are intended to be included within the scope of that term. A six month lifespan is generally sufficient for use in preventing restenosis. Shorter or longer periods, or permanent biostable materials may be appropriate for other applications. For post-surgical use in a ureter or bowel, implantation and necessary propulsive support may extend to a year. For complete dysfunction or frank replacement, the device may be made of permanent or near permanent materials.

The polycaprolactones disclosed in U.S. Pat. No. 4,702,917 to Schindler, incorporated herein by reference, are suitable bioabsorbable polymers for use in the devices and systems described herein. Polycaprolactones possess adequate mechanical strength being mostly crystalline even under quenching conditions. Despite their structural stability, polycaprolactones are much less rigid than the metals used in traditional stenting, thereby minimizing the risk of acute vessel wall damage from sharp or rough edges. In the case of a polycaprolactone, for example, the crystalline structure of the polymer generally maintain a constant outside diameter, when it is not subjected to the actuating forces or elements described herein.

The degradation process of polycaprolactone has been well characterized with the primary degradation product being nontoxic 6-hydroxy hexanoic acid of low acidity. Furthermore, the time over which biodegradation of polycaprolactone occurs can be adjusted through copolymerization.

Polycaprolactone has a crystalline melting point of 60° C. and can be deployed in vivo via a myriad of techniques which facilitate transient heating and varying degrees of mechanical deformation or application as dictated by individual situations.

Suitable biodegradable polymers for forming the walls include polyanhydrides. These materials frequently have fairly low glass transition temperatures, in some cases near normal body temperature, which makes them mechanically deformable with only a minimum of localized heating. Furthermore, they offer erosion times varying from several months to several years depending on particular polymer selected.

(c) Elastomeric Materials

The walls or one or more sections of the wall may contain one or more elastomers. Such construction will take advantage of stored forces achieved with expansion, twisting or propulsion of materials—followed by recoil of the material further propelling, adding to, or aiding the movement of a fluid through the lumen. This process is a mimic of a natural process seen in blood vessel known as the “Windkessel effect.” A wide range of elastomers may be utilized. For example, suitable elastomers include, unsaturated rubbers that can be cured by sulfur vulcanization, such as natural polyisoprene: cis-1,4-polyisoprene natural rubber (NR) and trans-1,4-polyisoprene gutta-percha; synthetic polyisoprene (IR for isoprene rubber); polybutadiene (BR for butadiene rubber); chloroprene rubber (CR), polychloroprene, Neoprene, Baypren etc.; butyl rubber (copolymer of isobutylene and isoprene, IIR); halogenated butyl rubbers (chloro butyl rubber: CIIR; bromo butyl rubber: BIIR); styrene-butadiene rubber (copolymer of styrene and butadiene, SBR); nitrile rubber (copolymer of butadiene and acrylonitrile, NBR), also called Buna N rubbers; or hydrogenated Nitrile Rubbers (HNBR) Therban and Zetpol (Unsaturated rubbers can also be cured by non-sulfur vulcanization if desired) For example, suitable elastomers also include, saturated rubbers that cannot be cured by sulfur vulcanization, such as EPM (ethylene propylene rubber, a copolymer of ethylene and propylene) and EPDM rubber (ethylene propylene diene rubber, a terpolymer of ethylene, propylene and a diene-component); epichlorohydrin rubber (ECO); polyacrylic rubber (ACM, ABR); silicone rubber (SI, Q, VMQ); fluorosilicone Rubber (FVMQ); fluoroelastomers (FKM, and FEPM) Viton, Tecnoflon, Fluorel, Aflas and Dai-El; perfluoroelastomers (FFKM) Tecnoflon PFR, Kalrez, Chemraz, Perlast; polyether block amides (PEBA); chlorosulfonated polyethylene (CSM), (Hypalon), or ethylene-vinyl acetate (EVA).

B. Materials or Elements to Alter Configuration of Tubular Structure

The device or system includes elements or materials that are able to alter the configuration of the tubular structure in a suitable time period and/or cycle to allow transport of fluids through the devices. In use, the devices are able to serve as local mini- or regional-pumps.

Propulsion of the fluids through the devices may occur via a range of elements that are either intrinsic to the construct material of the wall, contained within wall materials, or external to the wall of the device or within the endoluminal surface of the peripheral wall, or the endoluminal space in which the devices are located. Depending on the particular material or materials that form the walls or sections of the walls of the devices, are incorporated into the walls as elements, or that are included in a separate device or layer outside the peripheral wall of the device, different stimuli may be used to change the structure of the device from its resting state to an actuated state.

For example, active polymeric materials, which are thermo-sensitive, thermo-responsive, electro-conductive, acoustically sensitive and/or otherwise responsive to other external signals may be used to form the walls of the device. These materials alter their configuration when in contact with the relevant stimulus. Alternatively, one or more of these materials can be placed outside the walls of the device and apply a force on one or more sides of the device at one or more sections of the wall when they are in contact with the relevant stimulus.

By virtue of this process, coupled with imparting control and timing of this process, i.e. in a sequence over a length of material, they can impart a pumping function.

Suitable materials for forming one or more sections of the walls, all of part of the walls of the device, or for applying a force to the walls of the device are described below. Combinations of these materials, optionally with sections that are not responsive to the same stimulus (stimuli) may also be used.

a. Shape Memory Polymers

Shape-memory polymers (SMPs) are polymeric smart materials that are able to return from a deformed state (temporary shape) to their original (permanent) shape, after induced by a stimulus, such as temperature change, electric or magnetic field, light, or ultrasound.

SMPs can be characterized as phase segregated linear block co-polymers having a hard segment and a soft segment. The hard segment is typically crystalline, with a defined melting point, and the soft segment is typically amorphous, with a defined glass transition temperature. In some embodiments, however, the hard segment is amorphous and has a glass transition temperature rather than a melting point. In other embodiments, the soft segment is crystalline and has a melting point rather than a glass transition temperature. The melting point or glass transition temperature of the soft segment is substantially less than the melting point or glass transition temperature of the hard segment.

For example, U.S. Pat. No. 6,160,084 to Langer, et al., describes biodegradable shape memory polymers containing hard and soft segments, or at least one soft segment that is covalently or ionically crosslinked, or polymer blends, where the original shape of the polymer is recovered by a change in temperature or application of another stimulus. For example, at least one hard segment has a Tₜᵣₐₙₛ between −40 and 270° C., at least one soft segment has a Tₜᵣₐₙₛ at least 10° C. lower than that of the hard segment(s), which is linked to at least one hard segment, and at least one of the hard or soft segments includes a degradable region or at least one of the hard segment(s) is linked to at least one of the soft segment(s) through a degradable linkage.

A variety of different stimuli in addition to temperature can be used to change the shape of a device formed from a shape memory polymer from its original shape to a temporary shape.

i. Photochemical Stimuli

Photoreversible reactions can be used to link soft segments together and hold the polymer in a temporary shape. The original shape of a material is set by the hard segment. Upon photochemical cleavage of these linkages, the material returns to its original shape. As these reactions are photoreversible, the bonds can be made and broken through several cycles. However, each time the bonds are broken, they need to be remade in order to memorize the shape. Examples of such functional groups capable of undergoing photoreversible reactions are cinnamon acid derivatives and cinnamylidene acid derivatives. Linking and cleavage can be induced by different wavelengths of UV-light. In addition cleavage can occur during a thermal treatment.

In another embodiment, the polymers can include side chains with chromophores, such as azo- groups, that absorb light. The chromophores also may be incorporated into the main chain. The hard and/or soft segments can also include double bonds that shift from cis to trans isomers when the chromophores absorb light. Light can therefore be used to isomerize the segment, which can dramatically affect the Tₜᵣₐₙₛ of the segment. The original shape of such polymers is set by the hard segment. The polymer then can be deformed into a temporary shape. The temporary shape can be fixed by curing the polymer with light to cause photoisomerization. In this way, the polymer is hindered from returning to its original shape, because the thermal transition temperature has been increased. Solid to solid phase transitions also may be induced in this manner.

ii. Changes in Ionic Strength and/or pH

Various functional groups are known to crosslink in the presence of certain ions or in response to changes in pH. For example, calcium ions crosslink amine and alcohol groups, i.e., the amine groups on alginate can be crosslinked with calcium ions. Also, carboxylate and amine groups become charged species at certain pHs. When these species are charged, they can crosslink with ions of the opposite charge. The presence of groups which respond to changes in the concentration of an ionic species and/or to changes in pH on hard and/or soft segments results in reversible linkages between these segments. One can fix the shape of an object while crosslinking the segments.

After the shape has been deformed, alteration of the ionic concentration or pH can result in cleavage of the ionic interactions which formed the crosslinks between the segments, thereby relieving the strain caused by the deformation and thus returning the object to its original shape. Because ionic bonds are made and broken in this process, it can only be performed once. The bonds, however, can be re-formed by altering the ionic concentration and/or pH, so the process can be repeated as desired.

iii. Electric and Magnetic Fields

Various moieties, such as chromophores with a large number of delocalized electrons, increase in temperature in response to pulses of applied electric or magnetic fields as a result of the increased electron flow caused by the fields. After the materials increase in temperature, they can undergo temperature induced shape memory in the same manner as if the materials were heated directly. These compositions are particularly useful in biomedical applications where the direct application of heat to an implanted material may be difficult, but the application of an applied magnetic or electric field would only affect those molecules with the chromophore, and not heat the surrounding tissue.

iv. Ultrasound

Various shape memory materials contain reactive functional groups which fragment in response to applied ultrasound. Examples of these groups are those which form stable radicals, such as nitroso and triphenylmethane groups.

One can fix the shape of an object while forming bonds between two or more soft segments, for example by using heat or light. After the shape is deformed, the application of ultrasound can break the bonds between the soft segments, and relieve the strain caused by the deformation. The object will then return to its original shape. Because covalent bonds are made and broken in this process, it can only be performed once.

v. Temperature

Various shape-memory materials responsive to changes in temperature may be used to alter the configuration of tubular structure. These thermosensitive materials include crystalline thermoplastic resins showing crystallinity The thermally expandable materials expandable from a compressed state by heat may be obtained by a process comprising impregnating a form material comprising a crosslinked rubber with a crystalline thermoplastic resin (and optionally with a wax).

Examples of the crystalline thermoplastic resins include, but are not limited to, ethylene copolymers such as an ethylene-vinyl acetate copolymer, an ethylene-acrylic acid copolymer, an ethylene-vinyl alcohol copolymer, an ethylene-propylene copolymer resin and an ionomer resin; polyethylenes such as low-density polyethylene, intermediate-density polyethylene, high-density polyethylene and ultra-high-molecular-weight polyethylene; modified polyethylenes such as chlorinated polyethylene; polyester resins such as polyethylene terephthalate, polybutylene terephthalate, polycyclohexylenedimethylene terephthalate, a liquid crystalline polyester, poly-hydroxybutyrate, polycaprolactone, polyethylene adipate, polylactic acid, polybutylene succinate, polybutyl adipate and polyethylene succinate; polyketone resins such as polyether ether ketone; fluororesins such as polytetrafluoroethylene (a tetrafluoroethylene resin), a tetrafluoroethylene-perfluoroalkyl vinyl ether copolymer (a tetrafluoroethylene-perfluoroalkoxyethylene copolymer resin), a tetrafluoroethylene-hexafluoropropylene copolymer (a tetrafluoroethylene-hexafluoropropylene copolymer resin), a tetrafluoroethylene-ethylene copolymer (a tetrafluoroethylene-ethylene copolymer resin), polyvinylidene fluoride (a vinylidene fluoride resin), polychlorotrifluoroethylene (a chlorotrifluoroethylene resin), a chlorotrifluoroethylene-ethylene copolymer (a chlorotrifluoroethylene-eth-ylene copolymer resin) and polyvinyl fluoride (a vinyl fluoride resin); polyamide resins such as nylon 6, nylon 66, nylon 46, semi-aromatic nylon 6T, nylon MXD, nylon 610, nylon 612, nylon 11 and nylon 12; polypropylene resins such as atactic polypropylene, isotactic polypropylene and syndiotactic polypropylene; polyether resins such as polyethylene oxide and polypropylene oxide; and besides, polyacetal, isotactic polystyrene, syndiotactic polystyrene, a polyphenylene sulfide, a polyethernitrile, syndiotactic 1,2-polybutadiene, trans-1,4-polyisoprene, polymethyl-pentene, polyvinylidene chloride and polyvinyl alcohol, etc. Further, resins obtained by copolymerizing another monomer with these, or polymer alloys obtained by grafting another oligomer to these can also be used. Furthermore, a thermoplastic elastomer can also be used, and examples thereof include but are not limited to a polyolefinic thermoplastic elastomer, a polyester-based thermoplastic elastomer, a polyamide-based thermoplastic elastomer, a fluororesin-based elastomer.

Examples of waxes include animal waxes such as whale wax, bees wax, Chinese wax and wool wax; vegetable waxes such as candelilla wax, carnauba wax, Japan wax and sugar cane wax; mineral waxes such as montan wax, ozokerite, ceresin and lignite wax; synthetic hydrocarbon waxes such as Fischer-Tropsch wax and a derivative thereof, and low-molecular-weight polyethylene and a derivative thereof; modified waxes such as a montan wax derivative, a paraffin wax derivative and a microcrystalline wax derivative; aliphatic alcohols such as cetyl alcohol; fatty acids such as stearic acid; aliphatic esters such as glycerol stearate and polyethylene glycol stearate; hydrogenated waxes such as caster wax and opal wax; synthetic ketone amine amides such as armor wax and Acrawax; chlorinated hydrocarbons; synthetic animal waxes; and alpha-olefins.

Other examples of thermosensitive materials include expandable microspheres containing microscopic spheres having a thermoplastic shell encapsulating a low boiling point liquid hydrocarbon.

vi. Temperature- and/or Moisture-Sensitive Materials

Various shape-memory materials responsive to changes in temperature and/or moisture may be used to alter the configuration of tubular structure. Exemplary materials include ester-based thermoplastic polyurethane shape-memory polymers in the form of films, porous films, magnetic films, and wires as described in Huang, The Open Medical Devices Journal, 2:11-19 (2010). These materials show uniaxial tension at low temperature and shape recovery upon heating.

Other exemplary materials include humidity responsive self-bending bilayer-based actuators made by depositing layers composed of poly(N-isopropylacrylamide) -based microgels and the polyelectrolyte polydiallyldimethylammoniumchloride (pDADMAC) on a flexible substrate (Wei et al., Polym. Chem., 8:127-143 (2017)).

b. Electropolymeric Polymers

The device can be a smart biomedical tubular implant. Such devices typically contain one or more biologically compatible integrated electronic devices that provide for sensing, attenuation, communication, and/or power. Integrated electronic devices are preferably flexible, stretchable, or a combination of flexible and stretchable depending upon the demands of the application. Integrated electronic devices may maintain intimate conformal contact locally with the tissue of the organ or organ component. In some embodiments, the integrated electronic device is formed of a material that does not trigger an inflammatory response or that triggers a minimal inflammatory response. In some embodiments all or part of the integrated electronic device is encapsulated in a barrier material that does not trigger an inflammatory response or that triggers a minimal inflammatory response. Many biologically compatible coating or barrier materials are known, such as gold, platinum, SU-8, Teflon, polyglycerols, or hydrophilic polymers such as polyethylene glycol (PEG) or phosphorycholine, cell membranes or cell membrane-like material, aluminum oxide (Al₂O₃), hydroxyapatite (HA), silicon dioxide (SiO₂), titanium carbide (TiC), titanium nitride (TiN), titanium dioxide (TiO₂), zirconium dioxide (ZrO₂).

Representative electrical constructs that can be included in the integrated electronic devices include capacitors, inductors, antennas, interconnects (e.g. a conducting material wire), insulators, resistors (e.g. a potentiometer), transistors, diodes (e.g. light-emitting diodes), conducting materials/interconnects, edible printed circuit boards, electrodes, or piezoelectric materials.

i. Flexible Electronic Devices or Components

Flexible electronic devices or components of flexible electronic devices are in some embodiments formed of a material that is inherently flexible, i.e. a flexible organic polymer. Exemplary polymers include polyanilines, polycaprolactones, polylactic acids, copolymers and block-copolymers thereof. The polymers can be biocompatible or can be encapsulated with a biocompatible material. In some embodiments the flexible electronic devices or components of a flexible electronic device are fabricated from materials that are not inherently flexible but are fabricated sufficiently thin to provide the desired level of flexibility. The components of the flexible electronic devices may be fabricated for instance out of thin layers of crystalline silicon. The silicon layer may have a thickness less than or equal to 100 microns, optionally less than or equal to 10 microns, optionally less than or equal to 1 micron, optionally less than or equal to 500 nm. Flexible electronic devices may have a net bending stiffness less than or equal to 10⁸ GPa μm⁴, optionally less than 10⁷ GPa μum⁴, or optionally less than or equal to 10⁶ GPa μm⁴. The integrated electronic devices and components can be fashioned from conducting or semiconducting materials that are degradable, corrodible, or otherwise time-limited.

Integrated electronic devices and components can be made from magnesium, iron, silver, copper, tin, lead, actinide metals, lanthanide metals, alkali metals, alkaline-earth metals, noble metals, rare metals, rare-earth metals, or transition metals or alloys thereof. Integrated electronic devices and components can be made from a variety of materials and alloys such as those described in Ricker et al. (1994), “Corrosion of Metals” pgs. 669-728 in “Evaluation of Alternative In-Flight Fire Suppressants for Full-Scale Testing in Simulated Aircraft Engine Nacelles and Dry Bays. Section” edited by Grosshandler et al. NIST, 1994. The materials forming the integrated electronic device or component can be chosen based upon available rates of degradation or corrosion to choose the desired rate of degradation of the electronic device or component.

In some embodiments, electronic devices or components thereof are made flexible and/or stretchable by inclusion of a neutral mechanical surface to correspond to strain-sensitive layers or by selective use of strain isolation layers to isolate strain-sensitive layers from applied stresses and strains. In an example the electronic components or devices may reside in a neutral mechanical plane in a polymeric material or scaffold, where the surrounding material and/or layer contains a stretchable elastomer, such as for example a natural rubber, silicone rubber or polyurethane.

The devices can combine high quality electronic materials, such as aligned arrays of silicon nanoribbons and other inorganic nanomaterials; flexible and/or biodegradable electronic materials such as melanin; and ultrathin and elastomeric substrates, in multilayer neutral mechanical plane designs and with an optionally ‘wavy’ structural layout. The electronic devices may contain strain isolation layers that minimize or eliminate the influence of mechanical strain on device performance, thereby facilitating the use of such devices in a wide range of applications and of any arbitrary geometry. The integrated electronic devices may therefore be incorporated in shape-conforming biomedical implants without demonstrating strain-induced mechanical failures.

ii. Biodegradable Electronic Devices or Components

All or part of the components of the integrated electronic device can be biodegradable. The rate of degradation of all or some of the components can be adjusted to coincide with the useful life of the device. A wide range of biodegradable materials may be used in the integrated electronic device (e.g., distinct biodegradable materials may be used for each component), and the physical properties of the biodegradable materials may mirror those of materials that have been used in traditional organic thin-film microelectronic applications. However, unlike traditional organic thin-film microelectronic applications, in some embodiments, the active layer of the integrated electronic device contains a semiconducting material that is biodegradable, such as thin or ultra-thin silicon, a polymer, a protein, and/or a pigment (e.g., melanin). More specifically, the active layer of the biodegradable electronic device optionally contains a biodegradable, erodible or soluable semiconducting material, such as silicon, graphene, a polymer, a protein, carbon nanotubes, DNA, and/or an organic pigment.

For example, the biodegradable semiconducting material of the active layer may be silicon, graphene, carbon nanotubes, DNA or melanin. The biodegradable semiconducting material of the active layer also may have aromatic amino acids and their oligomers/polymers, porphyrin based proteins, block copolymers of synthetic conducting polymers if biodegradable blocks are sufficiently frequent to generate low molecular weight fragments, and metallized biopolymers.

The integrated electronic device may contain a biodegradable dielectric layer. The biodegradable dielectric lay may be silk, or poly(glycerol-sebacate) (“PGS”), which is a synthetic flexible biodegradable elastomer; polydioxanone; and/or poly(lactic-co-glycolic acid) (“PLGA”). Each of these materials has desirable mechanical properties and is biodegradable.

iii. Semiconductors

Use of the term “semiconductor” is consistent with this term in the art of microelectronics and electronic devices.

The polymeric material may include a semiconducting material, either as a formed electronic component or device, or as a constituent of a material forming an electronic component or device. Semiconducting materials include the range of elements and salts and/or oxides of these elements that may function as semiconductors including, but not limited to, silicon, germanium, gallium, boron, tin, lead, uranium, bismuth, barium, strontium, lithium, aluminum, indium, lanthanum, cadmium, copper, europium, platinum, nickel, mercury, silver, thallium, zinc. These materials may also be used singly or multiply as dopants. An example of doping includes DNA with admixed carbon, grapheme, or any of the listed semiconductor elements, their oxides or salts.

In some embodiments the semiconductor is an inorganic semiconductor. In some embodiments the semiconductor is an organic semiconductor. In some embodiments the semiconductor is a polymeric organic semiconductor. Useful inorganic semiconductors include those comprising element semiconductors, such as silicon, germanium and diamond, and compound semiconductors, such as group IV compound semiconductors such as SiC and SiGe, group III-V semiconductors such as AlSb, AIAs, Aln, AIP, BN, GaSb, GaAs, GaN, GaP, InSb, InAs, InN, and InP, group III-V ternary semiconductors alloys such as AlxGai.xAs, group II-VI semiconductors such as CsSe, CdS, CdTe, ZnO, ZnSe, ZnS, and ZnTe, group I-VII semiconductors CuCl, group IV-VI semiconductors such as PbS, PbTe and SnS, layer semiconductors such as Pb12, MoS2 and GaSe, oxide semiconductors such as CuO and Cu20. The term semiconductor includes intrinsic semiconductors and extrinsic semiconductors doped with one or more selected materials, including semiconductor having p-type doping materials and n-type doping materials, to provide beneficial electronic properties useful for a given application or device. The term semiconductor includes composite materials comprising a mixture of semiconductors and/or dopants. Specific semiconductor materials useful for in some embodiments include, but are not limited to, Si, Ge, SiC, AIP, AIAs, AlSb, GaN, GaP, GaAs, GaSb, InP, InAs, GaSb, InP, InAs, InSb, ZnO, ZnSe, ZnTe, CdS, CdSe, ZnSe, ZnTe, CdS, CdSe, CdTe, HgS, PbS, PbSe, PbTe, AIGaAs, AllnAs, AllnP, GaAsP, GalnAs, GalnP, AIGaAsSb, AIGalnP, and GalnAsP. Porous silicon semiconductor materials are useful for applications of aspects described herein in the field of sensors and light emitting materials, such as light emitting diodes (LEDs) and solid state lasers. Useful organic semiconductors include acenes, perylenes, fullerenes, phthalocyanines, oligothiophenes, and substituted derivatives thereof. Particular organic semiconductor compounds include sexithiophene, α,ω-dihexylsexithiophene, quinquethiophene, quaterthiophene, α,ω-dihexylquaterthiophene, α,ω-dihexylquinquethiophene, bis(dithienothiophene), anthradithiophene, dihexylanthradithiophene, polyacetylene, polythienylenevinylene, C60, [6,6]-phenyl-C61-butyric acid methyl ester, copper(II) hexadecafluorophthalocyanine, and N,N′-bis(pentadecafluoroheptylmethyl)naphthalene-1,4,5,8-tetracarboxylic diimide. Useful polymeric organic semiconductors include polyacetylenes, polydiacetylenes, polypyroles, polythiophenes, polyphenylenes, poly(arylene vinylenes), polyanilies, and copolymer and derivatives thereof. Particular polymeric organic semiconductors include poly(3-hexylthiophene), poly(phenylene vinylene), and poly(pyrrole). Organic semiconductors offer several advantages including inexpensive, easy shaping and manufacturing, a wide range of tunable properties via synthetic modifications, high degree of flexibility (especially in thin film devices), and their compatibility with a wide variety of substrates.

iv. Dielectric Elastomers

Dielectric elastomers (DEs) are smart material systems that produce large strains. They belong to the group of electroactive polymers (EAP). DE actuators (DEA) transform electric energy into mechanical work. They are lightweight and have a high elastic energy density.

v. Ferroelectric Polymers

Ferroelectric polymers are a group of crystalline polar polymers that are also ferroelectric, meaning that they maintain a permanent electric polarization that can be reversed, or switched, in an external electric field.

vi. Electroconstrictive Graft Polymers

Electroconstrictive graft polymers include a flexible backbone chain with branching side chains. The side chains on neighboring backbone polymers cross link and form crystal units. The backbone and side chain crystal units can then form polarized monomers, which contain atoms with partial charges and generate dipole moments. When an electrical field is applied, a force is applied to each partial charge and causes rotation of the whole polymer unit. This rotation causes electrostrictive strain and deformation of the polymer.

vii. Electrorheological Fluids

Electrorheological fluids change the viscosity of a solution with the application of an electric field. The fluid is a suspension of polymers in a low dielectric-constant liquid. With the application of a large electric field the viscosity of the suspension increases.

Electrorheological fluids can be contained within the device in a variety of configurations. For example, an electrorheological fluid may be in a wall layer, e.g., as a domain, or in an expandable and contractible bladder or other compartment, and when actuated, allows directional movement with net impingement on the device lumen, resulting in net contained fluid transport. These fluids may exist in small pillow-like domains, outpouching, “teardrop-like”, punching-bag shaped or polyp-shaped domains which when actuated allow net alteration (reduction) of the lumen volume temporarily and fluid propulsion. They may exist as twisted tubular structures—either parallel to or twisted—helical, coil or spiral shaped—encircling the device or within the wall or lumen of the device resulting in snake-like propulsion directionally of fluid upon actuation. The configuration may also be balloon shaped—round, tubular or otherwise.

viii. Ionic Polymer-Metal Composites

Ionic polymer-metal composites contain a thin ionomeric membrane with noble metal electrodes plated on its surface. These composites also have cations to balance the charge of the anions fixed to the polymer backbone. They are active actuators that show very high deformation at low applied voltage and show low impedance. Ionic polymer-metal composites work through electrostatic attraction between the cationic counter ions and the cathode of the applied electric field.

ix. Stimuli-Responsive Gels

Stimuli-responsive gels (hydrogels, when the swelling agent is an aqueous solution) are a kind of swellable polymer network with volume phase transition behavior. These materials change reversibly their volume, optical, mechanical and other properties by small alterations of certain physical (e.g. electric field, light, temperature) or chemical (concentrations) stimuli. The volume change of these materials occurs by swelling/shrinking and is diffusion-based. Gels provide the biggest change in volume of solid-state materials.

Also included are materials with contained coils, braids, wires or nets which can be electrically or magnetically actuated.

Another class of materials that can impart energy for transport include piezoelectric materials and acoustically active materials. These materials may form the walls or one or more sections of the walls of the devices and generate secondary waves and energy transfer which may be imparted to the fluid leading to transport via electricity or sound waves of as opposed to a purely mechanical, compressive or hydraulic forces.

x. Ultrasound-Sensitive, Ultrasound-Responsive, or Acusto-Amplifying Materials

The contained utlrasonic system or generator may itself impart a cavitation to the contained fluid. This imparting of force can lead to fluid propulsion. In another embodiment the device is constructed of acousto-sensitive, responsive or acusto-amplifying materials, such that external application of ultrasound will impart a force to the fluid within the device inside the organ or organ component.

c. Expandable Structures

An example of elements altering configuration of tubular structures are expandable structures formed with snapology methods. In snapology, interlocking strips of material snap together to create rigid structures. These expandable structure can be used on their own, or as building blocks to create other structures. One example of such structures are attenuated cubes, which have three degrees of articulation, are made of thin polymer sheets that fold flat but can also pop up in a variety of different ways and with pressure can inflate into a cube to create a bigger 3D structure. These materials may form nano-scale elements that may be inserted into devices, which, when placed into lumens may expand the expandable structures to alter the configuration of tubular structures.

d. Active Agents

One or more active agents can be included in the wall or in one or more layers of a multilayer wall, or as a coating on the outer and/or inner surface of the wall and released therefrom. Active agents may also be incorporated into the materials or elements altering the configuration of tubular structures.

The devices can include a coating to prevent biofilm growth, an antiseptic coating, etc.

The coating can be an antimicrobial coating, optionally containing one or more antibiotics or anti-bacterial agents, for example natural and synthetic penicillins and cephalosporins, sulphonamides, erythromycin, kanomycin, tetracycline, chloramphenicol, rifampicin and including gentamicin, ampicillin, benzypenicillin, benethamine penicillin, benzathine penicillin, phenethicillin, phenoxy-methyl penicillin, procaine penicillin, cloxacillin, flucloxacillin, methicillin sodium, amoxicillin, bacampicillin hydrochloride, ciclacillin, mezlocillin, pivampicillin, talampicillin hydrochloride, carfecillin sodium, piperacillin, ticarcillin, mecillinam, pirmecillinan, cefaclor, cefadroxil, cefotaxime, cefoxitin, cefsulodin sodium, ceftazidime, ceftizoxime, cefuroxime, cephalexin, cephalothin, cephamandole, cephazolin, cephradine, latamoxef disodium, aztreonam, chlortetracycline hydrochloride, clomocycline sodium, demeclocydine hydrochloride, doxycycline, lymecycline, minocycline, oxytetracycline, amikacin, framycetin sulphate, neomycin sulphate, netilmicin, tobramycin, colistin, sodium fusidate, polymyxin B sulphate, spectinomycin, vancomycin, calcium sulphaloxate, sulfametopyrazine, sulphadiazine, sulphadimidine, sulphaguanidine, sulphaurea, capreomycin, metronidazole, tinidazole, cinoxacin, ciprofloxacin, nitrofurantoin, hexamine, streptomycin, carbenicillin, colistimethate, polymyxin B, furazolidone, nalidixic acid, trimethoprim-sulfamethox-azole, clindamycin, lincomycin, cycloserine, isoniazid, ethambutol, ethionamide, pyrazinamide and the like; anti-fungal agents, for example miconazole, ketoconazole, itraconazole, fluconazole, amphotericin, flucytosine, griseofulvin, natamycin, nystatin, and the like; and anti-viral agents such as acyclovir, AZT, ddl, amantadine hydrochloride, inosine pranobex, vidarabine, and the like.

Optionally, the device includes other therapeutic or prophylactic agents, selected to treat or prevent a disease or disorder in the body, such as preventing infection, etc.

A variety of different active agents can be incorporated depending on the site of the organ or organ component and the disease or disorder in need of treatment or to be prevented.

The active agents may be incorporated in the materials or the elements altering the configuration of tubular structures. The active agents may be released once the material or element is actuated. Exemplary materials or elements with active agents include shape-memory polymers, electropolymeric polymers and expandable structures. Exemplary active agents include analgesics/antipyretics, antibiotics, antidiabetics, antifungal agents, antihypertensive agents, anti-inflammatories, antineoplastics, immunosuppressive agents, antimigraine agents, antianginal agents, antiarthritic agents, antigout agents, anticoagulants, thrombolytic agents, antifibrinolytic agents, hemorheologic agents, antiplatelet agents, antihistamines/antipruritics, agents useful for calcium regulation, antiviral agents, anti-infectives, steroidal compounds, hormones and hormone analogues, hypoglycemic agents, and hypolipidemic agents.

C. Controller

The devices or systems are able to control one or more properties associated with the cycle of changing the device from its resting state to its active state, such as the degree, frequency, timing, and/or synchrony. By varying one or more of these parameters, the rate of content or fluid transport, i.e. the flow rate, may be varied.

Additionally, the devices or systems are able to control the degree of shear and/or the extent of turbulence. Further, a range of sequences and/or a range of flow rates may be imparted for varying therapeutic and/or diagnostic purposes.

The control can be achieved by one or more controllers integrated in the device itself, or via other controllers implanted or inserted in the body or external to the body.

The controller can actuate the stimulus (or one or more stimuli) for the propulsive materials or elements. Optionally, the controller actuates the one or more materials or elements that are able to alter the configuration of the tubular structure in a suitable time period and/or cycle from the resting state to the actuated state to facilitate active transport of a fluid through the device. The controller also controls the intensity and frequency of the movements (e.g. twisting, contraction, expansion, etc) of the walls of the responsive materials to achieve the desired fluid flow profile, shear rate, and/or flow rate.

Optionally the device or system includes one or more sensors, which provide data to the controller, such as via controlled feedback, to allow the controller to modify one or more properties of the stimulus, or the cycle (e.g. intensity, frequency, etc) to achieve the desired fluid flow profile and direction.

One or more interconnects can connect two or more sections of the peripheral wall or different sections of the device. A controller located inside of the body, also referred to as an “intimate controller”, can be in electrical communication with the interconnects. An external controller can be in electrical communication with the interconnects and/or the controller located inside the body. Typically, the intimate controllers are in communication with the one or more sections of the device via the one or more interconnects in the device. The intimate controllers may control the sequence, direction and rate of actuation. The external controller may be used to turn on, turn off, monitor, adjust, and/or to control the sequence, direction, and/or rate of actuation of the device or one or more sections of the device. The intimate controllers may be in communication with the external controller to provide an overall control unit to control the sequence, direction and rate of actuation.

Any one of the controllers may be in communication with the one or more sensors in the device to provide feedback-controlled operability to the device. This typically allows the controller to modify one or more properties of the stimulus, or the cycle (e.g. intensity, frequency, etc) to achieve the desired fluid flow profile and direction.

The controllers may include a control circuitry, which may include hardware, software, firmware, or a combination thereof.

The interconnects, and the communication between the interconnects, intimate controller, and the external controller may be via wired connection or wireless or telemetric connection. The interconnects, the intimate controller located inside the patient's body, and the external controller are typically capable of transmitting a wireless control signal to each other to control or modify the sequence, direction, and rate of actuation.

II. Methods of Making the Devices

Devices can be fabricated using methods known in the art, such as patterning, photolithography, etching, molding, micromolding, three-dimensional printing (3D-printing), extrusion, hot pressing, or spray drying. Suitable methods for the manufacture of devices can be selected in view of a variety of factors, including the design of the device (e.g., the size of the device, the relative arrangement of device elements, etc.) and the component materials used to form the device.

Examples of suitable techniques that can be used, alone or in combination, for the fabrication of devices include LIGA (Lithographic Galvanoforming Abforming) techniques using X-ray lithography, high-aspect-ratio photolithography using a photoresist, microelectro-discharge machining (μEDM), high-aspect-ratio machining by deep reactive ion etching (DRIE), hot embossing, 3-dimensional printing, stereolithography, laser machining, ion beam machining, mechanical micro-cutting using micro-tools made of hard materials such as diamond, multi-layer physical vapor deposition, injection molding, extrusion, hot pressing, spray drying, and coating.

The devices, once formed, may be packaged for long term storage. The devices, once formed, may be lyophilized and packaged for long term storage.

### III. Methods for Using the Devices

A. Prepolymers

Devices may also be formed in place via imparting or importing construct materials via catheter or trocar system locally to the site of application, with an application means reconfiguring materials to create an endotubular structure. One or more devices, preferably a plurality of devices can be mixed with a liquid carrier and administered by spraying, brushing, rolling, or other application means or as a flowable liquid.

Examples include delivering to the desired site in the tubular organ pre-polymeric materials which may be polymerized in situ, such as described in U.S. Pat. Nos. 5,213,580 and 6,634,946 to Slepian as a gel paving means.

B. Preformed Device

Optionally, polymeric materials may be inserted percutaneously or surgically into the desired site in the patient as a constructed form, either as a tube or as a sheet, and may be reconfigured locally. Materials may be partially or fully reconfigured by virtue of unfurling, active expansion or modulation of structure by an energy means—e.g. heat expansion, acoustic/ultrasonic exposure, microwave, light irradiation—visible, UV, infrared or irradiation via other wavelengths.

The devices may be administered to a site in a patient via needles, trochars, sheaths or catheters,—i.e. hollow, low profile, small diameter access systems. Typically these devices range in diameter from 30 gauge-12 gauge (needles) or 3 french to 21 french (where 1 french=0.3 mm) (catheters and sheaths). However, the application devices may be even smaller.

The initial predeployment design and size of the device is determined by the specific application based upon the final deployed physical, physiological and pharmacological properties desired. Optionally, the device may be provided in the form of a rolled sheet of material, which is radially expanded and pressed into contact with a tissue surface by an unrolling procedure.

C. Uses

The materials and devices described herein can be used in all disciplines of medicine involving tubular organs and the flow of fluids there through.

The materials and devices described herein can be inserted into the tubular organ of a subject surgically or percutaneously. Subjects include a human and a veterinary animal.

One or more devices can be used in the same organ. For example, two or more devices can be inserted in a particular organ or organ segment in series or in parallel to restore, replace or augment, or otherwise modulate the deranged local transport function.

Tubular organs include the cardiovascular system, including the heart and blood vessels; the alimentary tract, including the esophagus, stomach, and small and large intestines; the respiratory system, including the trachea, bronchi, and alveoli; the lymphatic system; the genitourinary system with the kidney, ureter, bladder, and urethra; the reproductive system, including the fallopian tube, uterus or spermatic ducts; various glandular organs, including endocrine and exocrine tissues with ducts.

Examples of tubular organs include the cardiovascular system including the heart and blood vessels; the alimentary tract including the esophagus stomach small and large intestine; the respiratory tree including trachea, bronchi and alveoli; the lymphatic system; the genitourinary system with the hollow kidney, ureter, bladder, and urethra; the reproductive system, including the fallopian tubes, uterus or spermatic ducts; various glandular organs, including endocrine and exocrine tissues with ducts.

The tubular organs or organ component is generally on or a part of an organ that locally imparts the transport function of fluids. For example, the tubular organ to be treated can be the ureter. In the genitourinary system, the kidney is largely passive, filtering urine, providing a fluid and pressure head to the ureter, which has contained pulsatile and propulsive elements moving urine outward for elimination.

Other tubular organs in which the device(s) or system(s) can be placed include the gastrointestinal tract. In the gastrointestinal tract, contained motor function in the gut wall, in the form of peristalsis movement and undulation, is the primary mechanism by which food and its digestion and degradation products moves through the gastrointestinal system. This is in contrast to the circulatory system where blood vessels, while having some dynamism, such as in the form of spasm and constriction, are more passive in the sense that the heart provides the propulsive force moving blood throughout the more static circulatory system.

### IV. Exemplary Devices and Systems

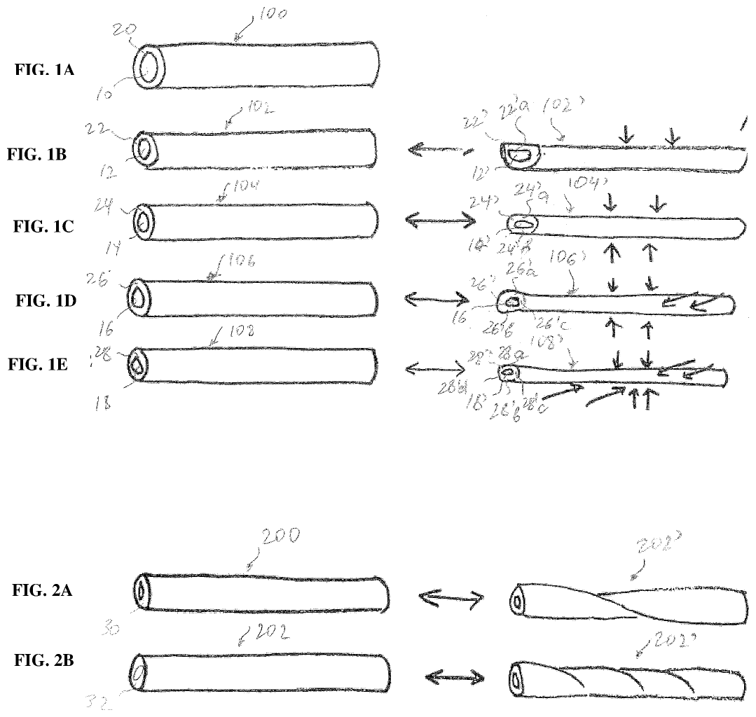

FIGS. 1A-5C depict a variety of different configurations for the tubular propulsion devices described herein.

The tubular propulsion devices can move fluids through a tubular organ by compression of one or more walls or portions of the walls in the device. For example, FIGS. 1A-1E show exemplary tubular propulsion devices 100 with lumen 10 and walls 20, and similar devices 102, 104, 106 and 108 in original, resting state (FIGS. 1A-1E) and four different configurations that the device can take in actuated state 102′, 104′, 106′, and 108′ (FIGS. 1B-1E). The respective device lumen 12, 14, 16, and 18 alters in configuration and constrict to lumen 12′, 14′, 16′, and 18′ when one or more portions of the device walls 22, 24, 26, and 28 move radially inward to form altered device walls, such as wall 22′ with one portion of the wall constricted (such as the top portion 22′a constricted), wall 24′ with two portions constricted (such as the top portion 24′a and the bottom portion 24′b constricted), wall 26′ with three portions constricted (such as the top portion 26′a, bottom portion 26′b, and front portion 26′c constricted), and wall 28′ with four portions constricted (such as the top portion 28′a, bottom portion 28′b, front portion 28′c, and the back portion 28′d constricted).

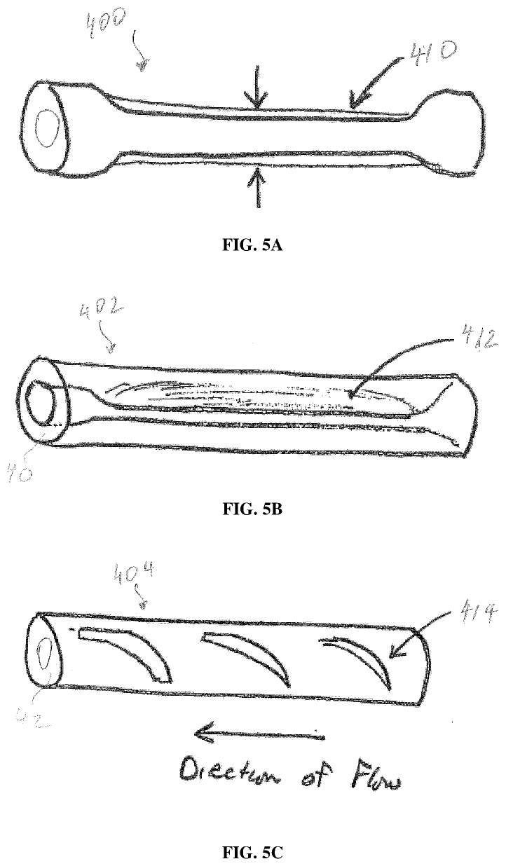

FIGS. 4A-4E are cross-sectional views of the devices illustrated in FIGS. 1A-1E comparing the cross-section of the device in its resting state (FIG. 4A) to the different cross-sections of the device in its actuated state (FIGS. 4B-4E). For example, following exposure to an appropriate stimulus, one portion, portion 22′a, of the peripheral wall 22 of the device 102 may be moved radially inward to form wall 22′ (FIGS. 1B, 4B). When the stimulus is removed, then the wall 22′ returns to its original shape (e.g. convex) (FIGS. 1B, 4B). Similarly, two portions, such as portions 24′a and 24′b of the peripheral wall 24 can be moved radially inward to form wall 24′ following exposure to an appropriate stimulus, and the wall 24′ can return to its original convex shape following removal of the stimulus (FIGS. 1C, 4C). Alternatively, three portions, such as portions 26′a, 26′b, and 26′c of the peripheral wall 26 can be moved radially inward to form wall 26′ following exposure to an appropriate stimulus, and the wall 26′ can return to its original convex shape following removal of the stimulus (FIGS. 1D, 4D). Optionally, four portions of the wall, such as portions 28′a, 28′b, 28′c, and 28′d of the peripheral wall 28 can be moved radially inward to form wall 28′ following exposure to an appropriate stimulus, and the wall 28′ can return to its original convex shape following removal of the stimulus (FIGS. 1E, 4E). More than four portions of the peripheral wall can be compressed at the same time or at different times, as needed, to facilitate movement of the fluid along the length of the device.

For a device in the shape of polygonal prism with more than four sides, optionally, more than four surfaces of the peripheral wall, up to the total number of sides of the polygon, can be compressed following exposure to an appropriate stimulus, and each of the surfaces of the wall can return to their original shape following removal of the stimulus. These processes can be repeated multiple times by exposing the device to the stimulus and removing the stimulus and in the desired time periods and cycles to move a fluid along the length of the organ or organ segment in which the device is placed.

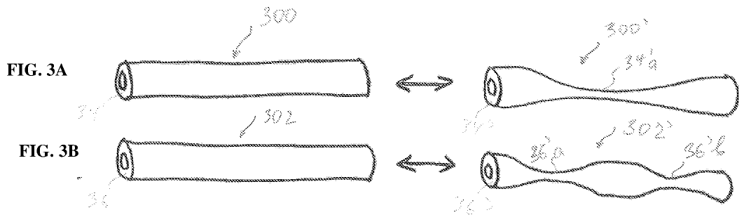

FIGS. 2A and 2B show the tubular propulsion devices 200 and 200 can move fluids through a tubular organ by twisting one time the wall 30 or more times the wall 32 in the devices 200 and 202 during actuation forming twisted devices 200′ and 202′ with twisted walls 30′ and 32′, respectively. For example, FIGS. 2A-2B are illustrations of exemplary tubular propulsion devices that push a fluid through the device via twisting actions. Optionally, when exposed to a stimulus, the wall of the device is actuated and twists one time, and then when the stimulus is removed, the wall returns to its original shape (FIG. 2A). Similarly, when exposed to a stimulus, the wall of the device is actuated and twists multiple times, and then when the stimulus is removed, the wall returns to its original shape (FIG. 2B). Each of the twists can occur simultaneously or at different times, such as sequentially, to aid in the movement of the fluid along the length of the device. These processes can be repeated multiple times by exposing the device to the stimulus and removing the stimulus and in the desired time periods and cycles to move a fluid along the length of the organ or organ segment in which the device is placed.

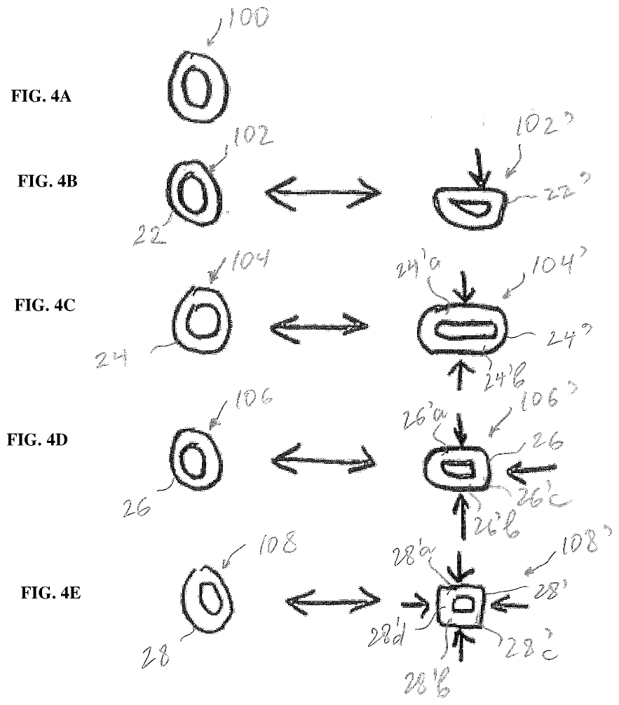

The tubular propulsion devices can move fluids through a tubular organ by contracting in one or more sections of the walls in the device. For example, FIGS. 3A-3B are illustrations of exemplary tubular propulsion devices 300 and 302 that push a fluid through the device via contractions in the walls 34 and 36 into contracted states 34′ and 36′ in the contracted devices 300′ and 302′ at device section 34′a, or device sections 36′a and 36′b, respectively. Optionally, when exposed to a stimulus, the wall of the device is actuated and contracts in one section as shown for wall 34′, and then when the stimulus is removed, the wall returns to its original shape (FIG. 3A). Similarly, when exposed to a stimulus, the wall of the device is actuated and can contract in two or more sections as shown for wall 36′, and then when the stimulus is removed, the wall returns to its original shape (FIG. 3B). Each of the sections of the wall can contract simultaneously or at different times, such as sequentially, to aid in the movement of the fluid along the length of the device. These processes can be repeated multiple times by exposing the device to the stimulus and removing the stimulus and in the desired time periods and cycles to move a fluid along the length of the organ or organ segment in which the device is placed.

As discussed above, different methods can be used to transition one or more walls of the tubular devices from an active (e.g. compressed, twisted, contracted, etc) state to its resting, original state repeatedly and thereby aid in the active transport of fluids through an organ or organ segment.

Each of the exemplary devices depicted in FIGS. 1A-7C, can be actuated externally, i.e. by one or more stimuli that act on another material or device that is separate from the device, or internally, i.e. by one or more stimuli that act on the walls, sections of the walls, or portions of the walls or portions of the sections of the walls. FIG. 5A shows the walls 410 of the tubular device 400 compressed via an external actuator (not shown). FIG. 5B shows a tubular device 402 inside of a tubular organ (not shown), where the device is compressed via one or more elements 412 in the walls 40 of the device itself. FIG. 5C shows a tubular device 404 where the walls 42 containing a plurality of flaps 414 that are actuated by any of the stimuli described above, and move from a resting position to a second position that pushes a fluid through the device. The device cycles from the resting position to the actuated (pushing) position multiple times and for a suitable period of time to push the fluid through the organ or organ segment in which the device is located.

A. Tubular Blood Pump

In some embodiments, the tubular devices described herein can be inserted into cardiovascular system to function as a blood pump. The blood pump is designed to apply a low shear force on the blood as it flows through the device.

A tubular blood pump can augment or replace a damaged heart or portions thereof. The pump has a tubular structure, as described above, with active contractile and propulsive elements within the walls of the device, or attached to the inside of the wall or the outside of the wall (e.g. a tube within a tube or multilayer device). To function as a blood pump, the walls of the device collapse (or contract) and expand (or return to their original shape) in a defined time period and cycle (see, e.g. FIGS. 1A-1E). Alternatively, the walls of the device twist one or more times and open up to their original shape in a defined period and cycle to move the blood through the device (see, e.g. FIGS. 2A-2B). Alternatively, the walls of the device move in a waving form, contracting in one section, that then returns to its original shape as the adjacent section contracts, which returns to its original shape as the next adjacent section (along the direction of blood flow) contracts, etc. with these wall motions repeated in a defined period and cycle to move the blood through the device (see, e.g. FIGS. 3A-3B). While these motions can be done directly to the wall, they can alternatively be forced on the walls by a second device or layer that is adjacent internally or externally to the device.

Optionally the walls contain electroactive polymers, electroactive wires, coils, or magnets to actuate the materials in the walls or surrounding the walls.

In another alternative device, a contained balloon everts, thereby pushing blood along the length of the tubular device.

For example, the device can be inserted inside of or parallel to the aorta to facilitate the active transport of oxygenated blood from the heart to the rest of the subject's body. Optionally, two devices can be inserted in series, such as with one acting as bypass for left-sided circulation, i.e. from the atria or ventricle to the aorta, or alternatively in the aorta and the other device similarly acting as bypass for right-sided circulation, i.e. from the atria or ventricle to the pulmonary artery. In this manner, the devices push deoxygenated blood to the lungs and push oxygenated blood from the lungs to the rest of the body, and thereby replace the heart, or augment the heart's function, as needed.

B. Ureter Pump

In some embodiments, the tubular devices described herein can be inserted into the genitourinary system, preferably into the ureter, to function as a ureter pump. The ureter pump is designed to provide pulsatile and propulsive forces to move urine outward for elimination.

A tubular ureter pump can augment or replace a damaged ureter or portions thereof. The pump has a tubular structure, as described above, with active contractile and/or propulsive elements within the walls of the device, or attached to the inside of the wall or the outside of the wall (e.g. a tube within a tube or multilayer device).

To function as a ureter pump, the peripheral wall(s) of the device collapse (or contract) and expand (or return to their original shape) in a defined time period and cycle (see, e.g. FIGS. 1A-1E). Alternatively, the peripheral wall(s) of the device twist one or more times and open up, i.e. untwist, to their original shape in a defined period and cycle to move urine through the device (see, e.g. FIGS. 2A-2B). Alternatively, the peripheral wall(s) of the device move in a waving form, contracting in one section, that then returns to its original shape as the adjacent section contracts, which returns to its original shape as the next adjacent section (along the direction of urine flow) contracts, etc. with these wall motions repeated in a defined period and cycle to move urine through the device (see, e.g. FIGS. 3A-3B). While these motions can be done directly to the wall, they can alternatively be forced on the walls by a second device or another layer that is adjacent internally or externally to the device.

Optionally the walls contain electroactive polymers, electroactive wires, coils, or magnets to actuate the materials in the walls or surrounding the walls.

In another alternative device, a contained balloon everts, thereby pushing urine along the length of the tubular device.

C. Multi-Section Propulsion Devices

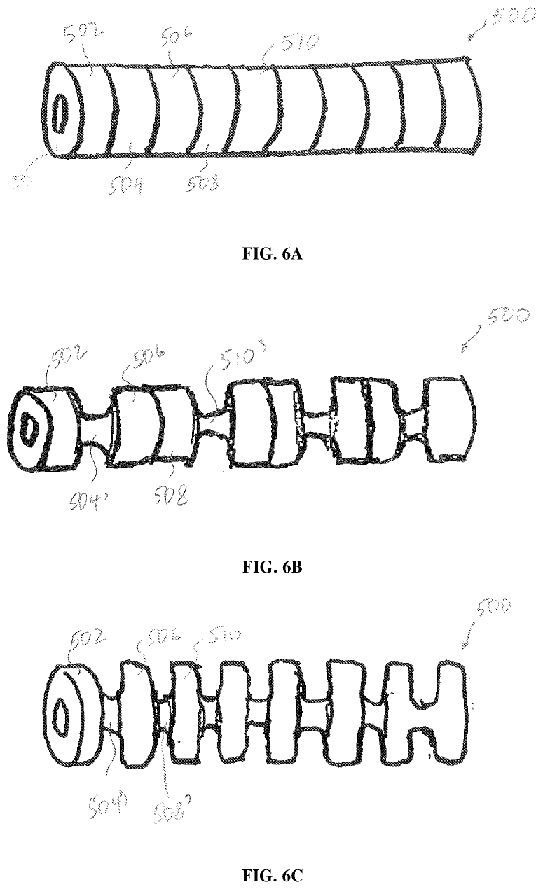

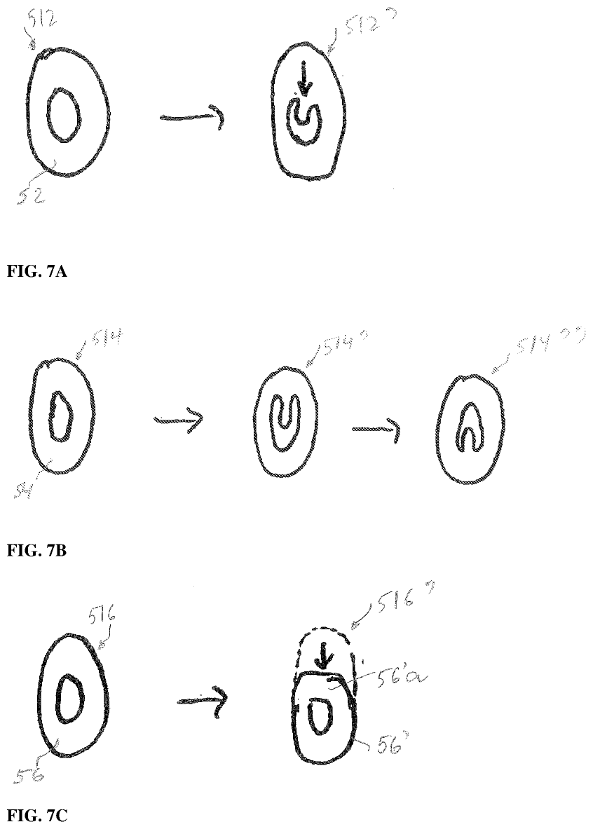

Exemplary embodiments of multi-section devices are shown in FIGS. 6A-7C.

An example of a multi-section propulsion device is a fluid propulsion device, such as a ureteral pump or stent.

In FIG. 6A a multi-section device 500 is shown. In this device, the peripheral walls 50 are fashioned so that the overall device is divided into and “in-series” arrangement of hollow circular or doughnut-shaped sections 502, 504, 506, 508, and 510, forming an overall device. Each section has a peripheral wall which allows either a portion of the peripheral wall to contract or narrow the lumen, or the doughnut has elements in the wall where the outer surface (i.e. outer perimeter of the peripheral wall) remains intact but aspects of the inner surface move radially inward, similarly leading to contracture or a limiting of a volume or area of the lumen. This “pulling-in” can occur in different amounts, or degrees of the overall circular arc of the construct. For instance, if one portion, such as an arc on the perimeter of the circular cross-section of the device and between two radii at 90° to each other pulls-in halfway the diameter of the lumen in the resting state, this results in about a 50% narrowing of the lumen (compared to the resting state). If a smaller portion, such as an arc between two radii at less than 90° to each other, such as at about 75°, about 60°, about 45°, about 30°, or about 15° to each other, moves radially inward, this will similarly create a smaller proportional lumen narrowing. All of this may be adjusted based on external programming using a controller, such as described below.

FIG. 6B shows a means by which this active, multi-section ureteral device 500 can be actuated with a defined sequence, allowing specific elements in the doughnut-shaped sections to be activated and alter the configuration of the section 504 into a radially-inward contracted state 504′ and the section 510 into a radially-inward contracted state 510′. If the sequence is actuated in a uni-directional manner this will induce directional lumen volume displacement of the contained fluid, in this case urine. It is understood that each part may have a valve or flap-like element, such as shown in FIG. 5C, to drive the direction of flow in a unidirectional fashion.

Another example of alternating element contraction of section 504 into a contracted state 504′ and section 508 into a contracted state 508′ is shown in FIG. 6C. Although specific examples of actuation are shown in FIGS. 6B and 6C, a wide range of sequence activation may be used.

FIGS. 7A-7C illustrate different configurations of a section containing one or more active wall elements in the section in their resting state and one or more actuated states . In FIG. 7A, an inner element of the wall 52 in section 512 moves radially inward forming an altered configuration section 512′. This may be accomplished via alternating magnet-like elements on opposing walls attached to a liner layer, allowing the nonporous inner lumen to move in a unidirectional fashion. Alternatively, magnetic materials may be in a given polymeric construct, e.g. via laminated thin films of magnetized material or interspersed magnetic particles, affording the same effect. Alternatively, shape-memory polymers, thermally expandable materials, thermal- and/or moisture expandable materials, or elements formed with snapology techniques may be in the peripheral wall or one or more sections or portions thereof. The particular elements and their location(s) within the peripheral wall or one or more sections thereof may be selected to achieve a similar, unidirectional volume change. Alternatively, air or fluid movements as described herein, may be utilized to provide directional movements encroaching the lumen and causing fluid shifts of the fluid within or flowing through the lumen.

FIG. 7B shows alternating elements in section 514, at opposing locations, such as 180° apart from each other, or located at other regular intervals along the perimeter of the walls, such as 15°, 30°, 45°, 90°, etc, or at irregular intervals along the perimeter of the walls. The location of the elements in the section may be utilized in walls 54 to sequentially create an activation scheme and alter the configuration of section 514 into 514′ or 514″, thereby displacing different zones of fluid.

FIG. 7C illustrates an alternative construction for altering the configuration of tubular structure when actuated, where the outer wall(s) 56 move(s) radially inward on one part of the wall, such as part 56′a, changing section 516 into altered configuration 516′ with wall 56′ where both the outer surface of a wall and the inner surface of the same wall move radially inward. Similarly, configurations depicted in FIGS. 4A-4E can be used in one or more sections, optionally in all of the sections, of a multi-sectional device to actuate the walls in the one or more sections of the device.

D. Exemplary Controllers

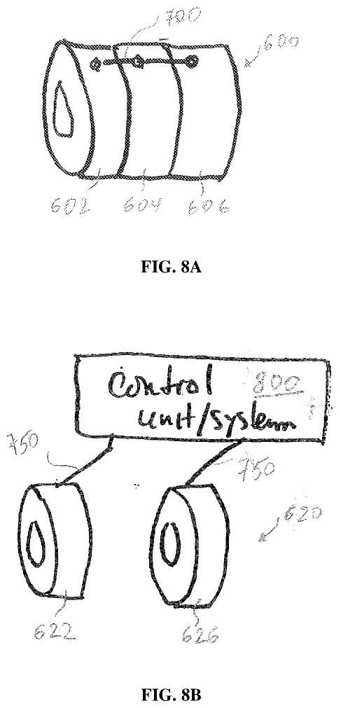

Examples of devices with controllers controlling the alteration of the configuration of the tubular structure of the devices are shown in FIGS. 8A-8C.

FIG. 8A shows the interconnects 700 between sections 602, 604, and 606 in the multi-section device 600. These may be direct communication connections. FIGS. 8B and 8C show an implanted or inserted, intimate controller 800 in contact with two sections 622 and 626 of a device 620 (FIG. 8B) via connections 750, or in contact with a plurality of sections 642, 644, 646, and 648, such as an entire device 640 (FIG. 8C) to control the sequence, direction and rate of actuation. FIG. 8C also illustrates a plurality of interconnects, e.g. 710, 720, 730, and 740, etc., where each interconnect connects two adjacent sections, e.g. sections 642 and 644, sections 642 and 646, sections 646 and 648, sections 648 and 650, etc. Further the device is in electrical communication with the implantable controller 800 via connections 750, and the implantable controller 800 is in electrical communication with an external controller 820 via connection 770. Together, the implantable controller 800 and the external controller 820 provide an overall control unit which is able to control the sequence, direction and/or rate of actuation of the tubular device 600. The external controller can be configured to turn on, turn off, monitor, adjust, and/or to control the sequence, direction and/or rate of actuation of the device. The connections 750 and 770 may be wired connections or wireless connections.

FIGS. 8A-8C illustrate a system for communication and intelligence for the pump/stent. Each section abutting or adjacent to another section may have a communication between the section. Shown in FIG. 8A are direct communication interconnects 700. This may occur via a direct configuration of a wire-like or flat sheet-like, or trace-like, communication or fiber optic means or conductive polymer or other means of information flow. This also may be accomplished by telemetric means and a wireless configuration.

FIG. 8B illustrates how different parts in the multi-section device, or between multiple sections as shown in FIG. 8C, communicate with an overall control unit to provide the intelligence and actuation sequence command and achieve sequential actuation, effective sequential pumping, direction control, and/or rate control for the flow of fluid through the device. The control unit may also receive information from sensors (not shown) contained in any part of the device. The information may relate to the pressure or flow or other parameters, e.g. temperature, pH or analyte concentration, such as Na, K, Cl, H₂CO₃, glucose, urea, bacterial, and/or protein presence or concentration.

The control system may also interact with response elements, which may also be configured to release one or more agents to modulate or mitigate a condition in the urine. For example, if a pump sensor detects calcium in the urine, it may direct the device to release pH altering chemicals contained therein to modify pH of the urine and reduce the potential for calcific deposition. Similarly, if bacteria are detected, the device or system can release an antimicrobial agent.

The control unit also maybe self-powered or maybe powered by fluid movement or via external power transmission wirelessly, such as with a transcutaneous energy transfer (TET) system. FIG. 8C shows an overall scheme in which a ureteral stent construct with a controller, suitable for implantation or insertion in the patient's body, may communicate to an external controller, allowing for turning on, turning off, monitoring, and/or other intelligent interaction with the system.

Although exemplified and described with respect to a multi-section device, the interconnects, connections, implantable controllers, and external controllers may be used with single tubular devices.

Those skilled in the art will recognize, or be able to ascertain using no more than routine experimentation, many equivalents to the specific embodiments of the invention described herein. Such equivalents are intended to be encompassed by the following claims.

1. A tubular propulsion device comprising a peripheral wall and a hollow center, and one or more materials or elements capable of inducing a temporary change in the structure of the peripheral wall of the device.

2. The tubular propulsion device of claim 1, wherein the temporary change is actuated by a stimulus selected from the group consisting of temperature change, electric field, magnetic field, ultrasound, light, and change in ionic solution, or a combination thereof.

3. The tubular propulsion device of claim 1, wherein the peripheral wall contains two or more sections, wherein at least one section is formed of a first material, and at least one different section is formed of a second material.

4. The tubular propulsion device of claim 1, wherein the device is a multilayer device, and wherein a first layer comprises the one or more materials or elements capable of inducing the temporary change in the structure of the peripheral wall of the device, and a second layer is stiffer than the first layer and does not contain the one or more materials or elements capable of inducing the temporary change in the structure of the peripheral wall of the device.

5. The tubular propulsion device of claim 4, wherein the first layer is around the second layer.

6. The tubular propulsion device of claim 4, wherein the first layer is inside the second layer.

7. The tubular propulsion device of claim 1, wherein the material or element capable of inducing the temporary change in the structure of the peripheral wall of the device is a shape memory polymer, a ferroelectric polymer, an electropolymeric material, an ionic polymer-metal composite, a stimuli-responsive gel, a piezoelectric material, an elastomer, or an acoustically active material, or a combination thereof.

8. The tubular propulsion device of claim 3, wherein the first material is capable of inducing a temporary change in the structure of the peripheral wall of the device, and wherein the second material is capable of inducing a temporary change in the structure of the peripheral wall of the device.

9. The tubular propulsion device of claim 8, wherein the first material responds to a first stimulus and the second material responds to a second stimulus.

10. The tubular propulsion device of claim 8, wherein the material is capable of inducing the temporary change in the structure of the peripheral wall of the device when subject to a stimulus, and wherein the second material does not change when subjected to the same stimulus.

11. The tubular propulsion device of claim 1, wherein the temporary change in the structure of the peripheral wall is a contraction of one or more surfaces of the peripheral wall, one or more twists in the peripheral wall, or one or more contractions in the peripheral wall.

12. The tubular propulsion device of claim 1, wherein the peripheral wall comprises one or more flaps, pores, or openings, or a combination thereof.

13. The tubular propulsion device of claim 1, further comprising a controller capable of actuating the one or more materials or elements capable of inducing the temporary change in the structure of the peripheral wall of the device.

14. The tubular propulsion device of claim 3, wherein each section is aligned with and attached to the adjacent section to form the tubular device.

15. The tubular propulsion device of claim 3, wherein one or more interconnects electrically connect each section with another section in the device.

16. The tubular propulsion device of claim 3, wherein a first interconnect electrically connects a first section with a second section.

17. The tubular propulsion device of claim 15, wherein the interconnects are in electrical communication with a controller capable of actuating the one or more materials or elements capable of inducing the temporary change in the structure of the peripheral wall of the device.

18. A method of augmenting, restoring, or replacing active transport of a fluid through a tubular organ or organ segment, comprising surgically or percuntaneously inserting into the organ or organ segment the device of claim 1.

19. A method of augmenting, restoring, or replacing active transport of a fluid through a tubular organ or organ segment, comprising percuntaneously inserting into the organ or organ segment a flowable material comprising prepolymers, and polymerizing the material to form the device of claim 1.

20-22. (canceled)

23. The method of claim 18, wherein the device further comprises a controller, and wherein the method further comprises the controller actuating the one or more materials or elements capable of inducing the temporary change in the structure of the peripheral wall of the device in a manner that directs the flow of a fluid.

24-25. (canceled)
